(19) United States
(12) Patent Application Publication
(10) Pub. No.: US 2012/0272955 A1
(43) Pub. Date: Nov. 1, 2012

(54) Automatic Tracheostomy Suctioning and Nebulizer Medication Delivery System

(76) Inventors: David Joseph Cool, Florissant, MO (US); Kenneth Nicholas Cool, Florissant, MO (US)

(21) Appl. No.: 13/458,405

(22) Filed: Apr. 27, 2012

Related U.S. Application Data
(60) Provisional application No. 61/480,771, filed on Apr. 29, 2011.

(51) Int. Cl. A61M 16/04; A61M 15/00; A61M 1/00; A61M 25/00; A61M 16/00; A61B 7/00

(52) U.S. Cl. 128/203.12; 128/204.18; 128/207.14

(57) ABSTRACT

Computer automated systems for suctioning tracheostomy patients, monitoring the physiological signs of a patient and administering a prescribed dose of medication to the tracheostomy patient.

### CROSS REFERENCE TO RELATED APPLICATION(S)

This Application claims the benefit of U.S. Provisional Patent Application Ser. No. 61/480,771, filed Apr. 29, 2011, the entire disclosure of which is incorporated herein by reference.

### BACKGROUND OF THE INVENTION

1. Field of the Invention

This disclosure is related to the field of automated systems for suctioning tracheostomy patients, monitoring the physiological data of patients, and administering a prescribed dose of medication to tracheostomy patients.

2. Description of Related Art

Among the oldest described surgical procedures, a tracheotomy consists of making an incision on the anterior aspect of the neck, thereby opening a direct airway to the respiratory system through an incision in the trachea. The resulting opening in the neck and trachea (known as a stoma) can serve independently as an airway or as a site for a tracheostomy tube (a generally curved tube) to be inserted. Accordingly, a tracheostomy allows a person to breathe without the use of his or her nose or mouth; i.e., air enters the respiratory system of the individual through the stoma or the tracheostomy tube rather than through the nose or mouth.

Tracheotomy procedures are performed on individuals who, for certain medical reasons, cannot breath on their own or have a difficult time breathing through the normal human respiratory passageways, the nose and mouth. Medical conditions and situations in which a tracheotomy is performed include, but are not limited to, severe facial trauma, head and neck cancers, large congenital tumors of the head and neck (e.g., a bronchial cleft cyst), acute angioedema and inflammation of the head and neck. Tracheotomies also are often utilized in the chronic setting where a patient has a need for long-term mechanical ventilation (e.g., comatose patients).

While a tracheostomy provides a patient who otherwise would have a difficult time breathing with a viable alternative passageway through which they can obtain air, the procedure does have some complications and drawbacks.

In the normal physiological framework in which an individual obtains air through the nose and/or mouth, in addition to functioning to supply air to and from the lungs, the upper airway warms, cleans and moistens air taken into the respiratory pathway. A tracheostomy bypasses these mechanisms. As a result, air entering the respiratory system of an individual through the stoma or a tracheostomy tube (also known as a “trach tube”) rather than through the upper airway is cooler, dryer and not as clean. In response to these changes to the way the air enters the respiratory system (cooler, drier and dirtier air), the body produces secretions.

The production of secretions by the body can cause two problems for a tracheostomy patient. First, secretions can become stuck in the trach tube or trach mask (i.e., the mist collar which attaches over the trach to provide moisture), thereby blocking the respiratory airway of the patient. Second, mucus and secretions in the trach tube or trach mask can become contaminated, which, in some cases, can lead to a chest infection. This is a particular problem for mechanically ventilated patients who are susceptible to ventilator associated pneumonia (VAP) from secretions which pool above the endotracheal (ET) tube cuff where they can contaminate the lower respiratory tract and cause infection.

Accordingly, suctioning mucus build-up from the trach tube, trach mask, ET tube cuff and other artificial airways known to those of ordinary skill in the art and associated with tracheostomy and ventilator patients is important to prevent a secretion plug from blocking the airway, stopping the patient's breathing and to prevent contamination and subsequent infection. Generally, suctioning of the trach tube, trach mask and ET tube cuff is performed by health care practitioners in a hospital setting. However, in many instances of at-home long term care, the duty of suctioning a patient falls upon the shoulders of the patient's family and loved ones.

Generally, a loved one or medical practitioner taking care of a patient is instructed to suction the patient's airway: 1) any time the patient feels or someone can hear mucus rattling in the tube or airway; 2) in the morning when the patient first wakes up; 3) when there is an increased respiratory rate (i.e., when the patient is working hard to breathe); 4) before meals; 5) before going outdoors; and 6) before going to sleep. Currently, a patient's airway is generally suctioned through the connection of a suction catheter (which is attached to a suctioning machine) to the patient's airway to remove any secretion build up in the airway. Accordingly, anyone caring for a patient on a trach tube or ventilator must be constantly vigilant, looking for indications of secretion build-up (such as an increased heart rate) to maintain the comfort of the patient, prevent infection and prevent secretions from blocking the airway.

In addition, the suctioning procedure has many complications and many patients find the experience painful and anxiety inducing. Major complications from the process include: hypoxia related to an interruption in inspired oxygen flow and partial airway obstruction as the catheter passes into the tracheostomy, trauma and infection.

Thus, the maintenance of the airway of a tracheostomy or ventilator patient can be a burden both for the patient and his or her caregiver. For the patient, there is a constant stress and fear of drowning as secretions slowly build in the airway. Further, the suctioning process can result in major complications and introduce infection. For the caregiver, it can be stressful to constantly monitor the airway for secretions. There can also be a financial and emotional burden. Further, due in part to the manual manipulation of the suctioning apparatus, the current suctioning practices can be a source of contamination. Accordingly, there is a need in the art for a device to automatically suction the airway of a tracheostomy and/or ventilator patient at the proper time and instances, eliminating the need for manual suctioning by a caregiver.

Further, in the medical field there is often a need to monitor the vital signs and other physiological data of patients. Generally, in the modern practice of medical monitoring the physiological data of a monitored patient is displayed on a screen or other commonly utilized user interface. The physiological data can be displayed in a number of ways including continuous data channels along a time axis and computer parameters such as the maximum, minimum and average values and pulse and respiratory frequencies. One method of medical monitoring used in the art is digital signal processing technology which has the advantages of miniaturization, portability, and multi-parameter monitoring that can track many different vital signs at once. Vital signs of a patient which are often monitored include: the patient's pulse and blood oxygenation levels (often via pulse oximetry); heartbeat (via a transthoracic interpretation of the electrical activity of the heart over a period of time as detected by electrodes attached to the outer surface of the skin which detect and amplify the tiny electrical changes on the skin that are caused when the heart muscle depolarizes during each heartbeat); blood pressure (invasively through an inserted blood transducer assembly or noninvasively with an inflatable blood pressure cuff); body temperature (often through a thermoelectric transducer); cardiac output (often via pulmonary artery catherization); and respiration rate (often via a thoracic transducer belt or an electrocardiograph), among others.

Generally these vital signs are displayed on a medical monitor with an integrated display that displays the data in real time. Certain medical monitors also have the ability to transmit data to a network or other methodology known to those of ordinary skill in the art for processing, storing and displaying information. One issue with this current form of medical monitoring is its generally episodic nature; the present vital signs of a patient are shown, however comprehensive and advanced computerized medical diagnostic interfaces are rarely a part of traditional medical monitoring. Continuous data collection, analysis and presentation of a patient's changing physiological state over time can reveal patterns and offer medical caregivers a comprehensive dataset of a patient's progression over time from which to make an informed decision. For example, human blood pressure fluctuates throughout the day. Episodic measuring only gives isolated data and may miss the peak and trough points that deserve a physician's attention for cardiovascular care. Accordingly, there is also a need in the art for an integrated monitoring system and interface that is able to compile, analyze and present continuous physiological data of a patient over time to a medical caregiver.

### SUMMARY OF THE INVENTION

Because of these and other problems in the art, described herein, among other things, are computer automated systems for suctioning tracheostomy patients, monitoring the physiological signs of a patient and administering a prescribed dose of medication to the tracheostomy patient.

In one embodiment, the tracheostomy apparatus comprises: an air column having a proximal and a distal end and a length there between, the proximal end being inserted into the tracheostomy opening of a patient and the distal end being connected to an oxygen supply line; and at least one audio device connected to the length of air tubing; wherein the at least one audio device receives audio frequencies including the patient's breath from the air tubing; and wherein a processor interprets and responds to said audio frequencies.

In one embodiment the tracheostomy apparatus of claim 1 further comprises a length of suctioning catheter having a proximal end and a distal end, the proximal end being inserted into the tracheostomy opening of a patient and the distal end being attached to a suction pump.

In one embodiment of the tracheostomy apparatus the at least one audio device is capable of receiving frequencies from about 30 Hz to about 3,000 Hz. Generally at least one audio device is chosen from the group consisting of: microphones, transducers and speakers.

In yet another embodiment, the tracheostomy apparatus will further comprise at least one nebulizer connected to the air column.

Also disclosed herein is a non-transitory computer readable medium comprising: computer readable instructions for counting down a pre-set timing mechanism; and computer readable instructions for triggering the suctioning of an air column of a patient and the delivering of a nebulizing dose of medication by turning on and off a suction pump and a nebulizer in a sequence of pre-set time on and rest cycles.

Also disclosed herein is a non-transitory computer readable medium comprising: computer readable instructions for determining when noise detected within an air column of a patient has reached a certain pre-set level; computer readable instructions for triggering the suctioning of the air column of a patient for a set period of time when the noise reaches the pre-set level. In one embodiment this non-transitory computer readable medium will further comprise: computer readable instructions for triggering the continued suctioning of a patient at the end of the set period of time until the noise detected is below the certain pre-set level.

Also disclosed herein is a method of automatically suctioning a patient, the method comprising: setting a pre-set timing mechanism with certain pre-defined intervals; counting down the pre-set timing mechanism; and triggering the suctioning of a patient for a pre-set period of time when a certain pre-defined interval is reached.

Finally disclosed herein is a method of automatically suctioning a patient, the method comprising: determining the level of noise within an air column to a patient; determining when the level of noise within the air column reaches a certain pre-defined level; triggering the suctioning of the air column for a pre-set period of time when the noise reaches the certain pre-defined level; determining if the noise is still at the certain pre-defined level at the end of the pre-set suctioning time period; and continuing the suctioning of the air column for another pre-set period of time if the noise is still at the certain pre-defined level.

### BRIEF DESCRIPTION OF THE DRAWINGS

FIG. 1 provides a diagram of the suctioning methodology of the prior art.

FIG. 2 provides a perspective view of an embodiment of the suctioning/tracheostomy apparatus component of this system.

FIG. 3 provides a perspective view of an embodiment of the suctioning/tracheostomy apparatus component of the system in which the suctioning catheter is inserted into the stoma of a patient at the same time as the air tubing.

FIGS. 4a-b provides an embodiment of the connection of the audio device to the air tubing in an embodiment of the suctioning/tracheostomy apparatus.

FIG. 5 provides a flow chart of an embodiment of the computer automated system for suctioning a tracheostomy patient which incorporates the Auto Sequence Mode, the On Demand Mode and the Manual Mode.

FIG. 6 provides a flow chart of an embodiment of the On Demand Mode.

FIG. 7 provides a flow chart of an embodiment of the Auto Sequence Mode.

FIG. 8 provides a depiction of an embodiment of the Monitor Interface.

FIGS. 9a-b provides a depiction of an embodiment of the Informational Interface.

FIGS. 10a-d provides a depiction of an embodiment of the History Interface.

FIGS. 11a-b provides a depiction of an embodiment of the Setup Interface.

FIGS. 12a-b provides a depiction of an embodiment of the Recording Interface.

### DESCRIPTION OF THE PREFERRED EMBODIMENTS

As a preliminary matter, it is noted that, throughout this disclosure, the term “computer” will be used to describe hardware which implements functionality of various systems. The term “computer” is not intended to be limited to any type of computing device but is intended to be inclusive of all computational devices including, but not limited to, processing devices or processors, personal computers, work stations, servers, clients, portable computers, smartphones, tablets and hand held computers. Further, each computer discussed herein is necessarily an abstraction of a single machine. It is known to those of ordinary skill in the art that the functionality of any single computer may be spread across a number of individual machines. Therefore, a computer, as used herein, can refer both to a single standalone machine or to a number of integrated (e.g., networked) machines which work together to perform the actions. In this way the functionality of a computer may be at a single computer or it may be a network whereby the functions are distributed. Further, the term “software” refers to code objects, logic, or command structures, written in any language and executable in any environment designed to be executed by or on a computer. It should be recognized that software functionality can be hardwired onto a chip or into other hardware while still considering it software within the meaning of this disclosure.

Disclosed herein, among other things, are devices, systems and methods for automatically suctioning, monitoring and delivering a prescribed dose of medication to a patient through the real-time monitoring and interpretation of noises in a patient's airway. As an initial matter, a high level overview of possible individual components of the devices, systems, and methods in various embodiments, including the hardware and software components and the components of the suctioning/nebulizing apparatus, will be discussed.

In general, the computer automated systems for suctioning a patient, monitoring a patient and administering a prescribed dose of medication to a patient described herein, in varying embodiments, is comprised of several components. One contemplated component of the system is a computer known to those of ordinary skill in the art. In one embodiment, the computer hosts the human-machine interface which also will be referred to herein as the user interface. In other embodiments, it is also contemplated that the computer will host a database for the system. In general, the user interface provides the following functionality for the computer automated systems described herein. First, as will be further described herein, it may provide a display of current information and data, both episodic and continuous, regarding a patient's physiological information and data. Second, it may provide an interface for a user to interact with the database for the system. The database for the system is generally a database where data compiled by the system is stored. In one embodiment, all of the data compiled by the system outside of the stored audio files is stored in the database. Information stored and accessible to the user in the database includes, but is not limited to, operator inputs, historical records (such as records for the suction pump, nebulizer, system start/shutdown and user interface connect/disconnect), patient information, doctor information, nurse information, medication information, supplies information and help information. Third, it may provide a means of communication to and from the real-time controller. Fourth, it provides a mechanism for recording and playing back stored audio files. Finally, in different embodiments, the interface may provide other functionalities which will be described in more detail later in this application which may include patient information, doctor information, nurse information, medication information, supply information and help information.

Another contemplated component of the computer automated systems for suctioning a patient, monitoring a patient and administering a prescribed dose of medication to a patient described herein in certain embodiments is the real-time controller. Generally, the real-time controller is a computer known to those of ordinary skill in the art. In one embodiment, it is contemplated that the real-time controller and the personal computer will comprise a singular piece of hardware. The real-time controller functions to host the real-time application software. Generally, in one embodiment, as long as the system has some source of power, it is contemplated that the real-time application software will be “on” and functional. The responsibilities of the real-time application software of the system include some of the primary operations involved in patient care facilitated through the system such as suctioning and nebulizing, which will be described in further detail herein. Other contemplated functions for the real-time application software may include, but are not limited to, communication to and from the user interface, communication to and from the field-programmable gate array processor, maintenance of a persistent file containing setup parameters, maintenance and managing of a log file containing performed machine operations, and maintenance and managing a log file containing operator changes to the setup parameters for the system. It is contemplated that the real time controller will be networked to the personal computer and other components of the system in a manner of networking known to those of ordinary skill in the art. In addition, it is also contemplated that the real time controller may be integrated to the interface of the field-programmable gate array processor. Notably, any real-time controller known to those of ordinary skill in the art such as, but not limited to, National Instrument's cRIO-9073, 266 MHz Real-Time Controller is contemplated as the component hardware of this portion of the system.

Another component of the computer automated systems for suctioning a patient, monitoring a patient, and administering a prescribed dose of medication to a patient described herein is the field-programmable gate array processor. In one embodiment, the field-programmable gate array processor will be integrated to and networked with the real-time controller, personal computer interface, and other components of the system. Notably, any processor known to those of ordinary skill in the art, such as the National Instruments cRIO-9073 processor, is contemplated for this hardware component of the system. In one embodiment, the field-programmable gate array processor may host the field-programmable gate array portion of the software application of the system.

The field-programmable gate array portion of the software application of the system may be responsible for the following functions of the system: communication to and from the real-time controller; acquiring data from the microphone and other sensors; outputting data to control the external equipment and software of the system, such as the suction pump, nebulizer and audio output; signal conditioning to remove ambient noise sources from the patient signal, including but not limited to, digital filtering, active noise cancellation, linear regression analysis, principal component analysis or proper orthogonal decomposition; acoustical analysis of patient signal (including sound level analysis, fractional-octave analysis, breath rate analysis, and other mathematical algorithms known to those of ordinary skill in the art to create unique metric data related to patient airway status) and performing other mathematical algorithms known to those of ordinary skill in the art for which the field-programmable gate array portion is uniquely suited.

Similar to the real-time application software, the field-programmable gate array software includes features which are designed to be always on as long as the unit has power. It is also contemplated that, in certain embodiments, the field-programmable gate array software is uniquely suited for features designed to run at extremely high rates with high computational speed and efficiency and without the possibility of operating system interruption.

Another contemplated component of the computer automated systems for suctioning a patient, monitoring a patient and administering a prescribed dose of medication to a patient described herein is the audio device and the audio device input module. Generally, any microphone or other similar audio monitoring tool which can be mounted to the air column of a patient as described herein to listen to a patient's breathing and other noises in the air column is contemplated as the audio device. In one embodiment, the audio device may be connected to the rest of the system via an audio device input module known to those of ordinary skill in the art, such as the National Instruments NI-9234 Analog Input Module. In one embodiment, this module may be interfaced to the field-programmable gate array processor. However, a method known to those of ordinary skill in the art for connecting the audio device to the rest of the system such that the audio frequencies in the air column received by the audio device may be interpreted by the system is contemplated.

Another contemplated component in certain embodiments of the computer automated systems for suctioning a patient, monitoring a patient and administering a prescribed dose of medication to a patient described herein is the analog output module. Generally, this component of the system is the external connection of the system to a sound system with live audio. In one embodiment, it is contemplated that this component of the system will be interfaced to the field-programmable gate array processor for the sourcing of the audio data captured by the system such as the breathing sounds of the patient. Generally, any analog output module or other methodology for connecting the system to an exterior audio output system such as a stereo known to those of ordinary skill in the art, such as National Instruments NI-9263 Analog Output Module, is contemplated as the analog output module of this application.

In certain embodiments of the computer automated systems for suctioning a patient, monitoring a patient and administering a prescribed dose of medication to a patient described herein, one or more digital output modules (or other similar signal relate for connecting the various hardware devices and other components of the system) is another contemplated component. In one embodiment, the digital output module may field wiring from the front of the module to the external relays and is interfaced with the field-programmable gate array processor for control.

The suction pump is another contemplated component in the computer automated system for suctioning a patient, monitoring a patient and administering a prescribed dose of medication to a patient described herein. Generally any suction pump apparatus known to those of ordinary skill in the art that is capable of removing secretions and excess fluids from a patient's airway is contemplated in this disclosure. In one embodiment, the suction pump will be connected to the system via the digital output module by way of an external electronic relay. However, any method known to those of ordinary skill in the art for connecting the suction pump to the system is generally contemplated.

The nebulizer is another contemplated component of the computer automated system for suctioning a patient, monitoring a patient and administering a prescribed dose of medication to a patient described herein. A nebulizer, as that term is used herein, shall include any device known to those of ordinary skill in the art for turning a liquid or gaseous medication into smaller particles to be delivered into a patient's respiratory system. Generally any nebulizer or other analogous device known to those of ordinary skill in the art for moistening and medicating a patient's airway is contemplated. In one embodiment, the nebulizer may be connected to the system via connection to the digital output module by way of the external relay. However, it should be understood that any method known to those of ordinary skill in the art for connecting a nebulizer to the system is contemplated.

Another component of the computer automated system for suctioning a patient, monitoring a patient and administering a prescribed dose of medication to a patient described herein is the air tubing. Air tubing includes any air tubing or hose, or associated device known to those of ordinary skill in the art that is placed into a patient's trachea to provide an airway or serve as a conduit for the administration of certain drugs. Examples of contemplated air tubing include, but are not limited to, corrugated blue or clear air tubing or hosing. Air tubing shall also include devices associated with providing air, medicine or other deliverable to a trached or ventilated patient, which could collect secretions. Such devices include, but are not limited to, trach tubes, trach masks, ET tube cuffs, artificial noses (caps that can be attached to a trach tube to help maintain humidity), aerosol tubing (tubing used to deliver inhaled medicine), cannula, and Passy-Muir speaking valves. This broad category of tubing, airways and devices shall be collectively referred to herein as the air column.

Simplified, the computer automated system for suctioning a patient, monitoring a patient and administering a prescribed dose of medication to a patient described herein monitors the secretion buildup in the airway of a patient and the vital signs of a patient through audio technology—the breath patterns of a patient are monitored by measuring the resonate audio frequencies of a trached individual's breath in an air column. Generally secretion buildup in the air column alters the normal audio frequencies in the air column caused by a patient's breathing. As secretion builds up in the airway, a patient's breathing generally gets rasped and loud. Once the audio frequencies reach a certain pre-defined level (a level at which the secretion started to build in the patient's airway causing impedance to their breathing), the suction pump is automatically triggered to begin automatic suctioning of the airway without interruption of the patient's air supply. Further, trached patients often suffer from constriction of the airway caused by various reasons (e.g., the underlying patient condition, COPD, emphysema, asthma and the trach tube itself). Airway construction of a patient also results in labored and loader breathing. Nebulized bronchodilators are often used to treat airway constriction. The disclosed system also monitors the breathing sounds of an individual to determine when the audio frequencies reach a level that signifies the belabored breathing brought about by airway construction. Once this level is reached, the suction pump and/or the nebulizer is automatically triggered. This system of audio monitoring allows for real-time, non-invasive monitoring of a patient's airway, detection of any fluid-type obstructions and removal of any fluid-type obstructions by activation of a suction pump as needed without the assistance of a caregiver. Accordingly, the manual monitoring and suctioning of the present art is no longer required. Notably, at various times within this application the patient shall be referred to as a tracheostomy patient or a ventilator patient. It shall be understood that these terms are intended to be descriptive and not limiting—any patient with an air column and potential for the build-up of secretions within the air column is contemplated as a patient.

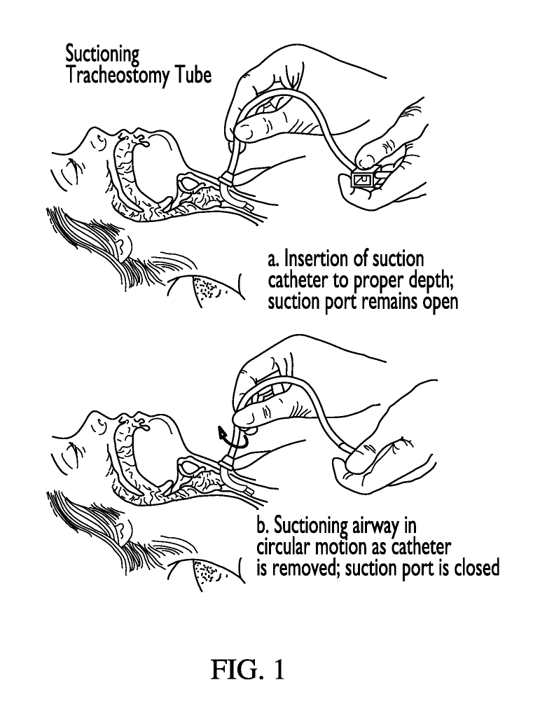

As touched upon previously, one aspect of the computer automated system for suctioning a patient, monitoring a patient, and administering a prescribed dose of medication to a patient described herein is the suctioning/tracheostomy apparatus which is inserted into a patient's tracheostomy stoma. In the traditional methodologies, suctioning occurs through a manual process. First, an individual may remove the trach tube providing air to the patient through the patient's tracheostomy stoma. Next, an individual places a suctioning catheter which is connected to a suction pump into the patient's tracheostomy stoma. Once inserted within the tracheostomy stoma, suction is applied and the suction catheter is rotated in the tracheostomy stoma and, the patient's airway, the trach cuff and other problematic areas in the air column to remove mucus and other secretions which can inhibit breathing. Suctioning generally occurs for no longer than ten (10) seconds. Then the suction catheter is removed and the patient is allowed to rest. This process is continued until the mucus and other secretions are removed from the patient's airway. A diagram of this suctioning methodology of the prior art is provided in FIG. 1.

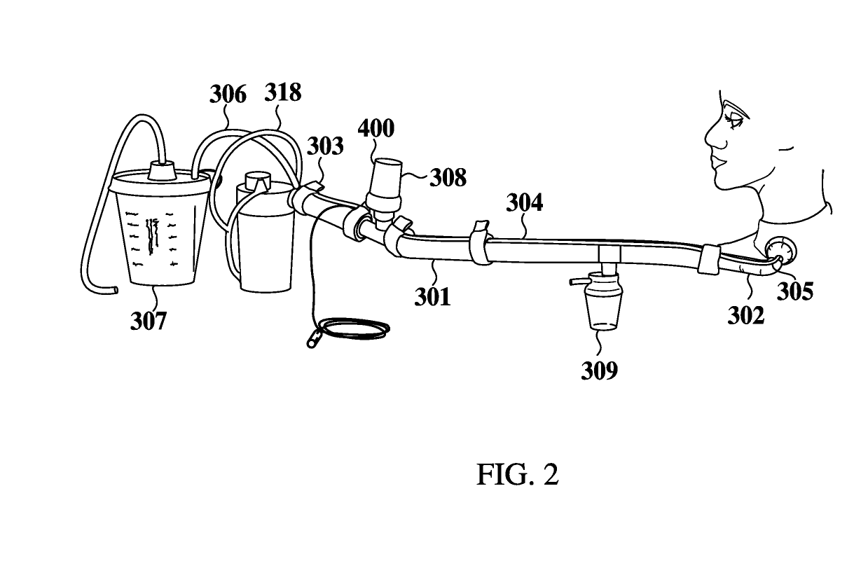

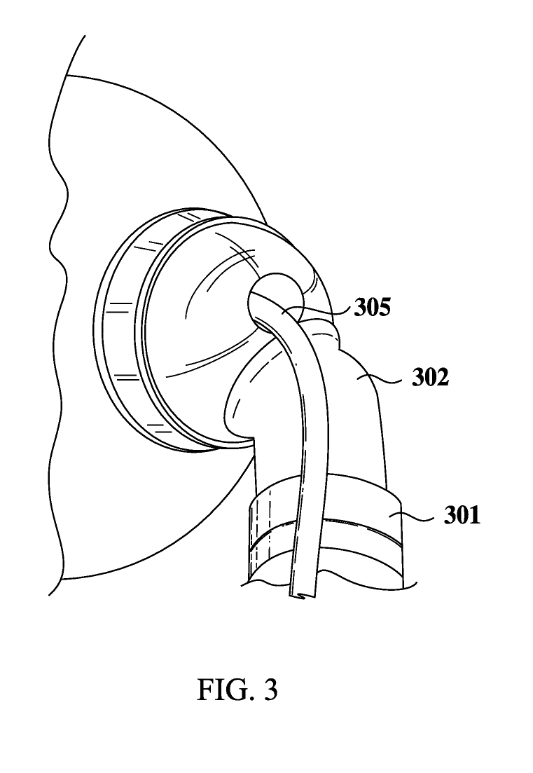

The computer automated system for suctioning a patient described herein eliminates the need for this manual suctioning process. An embodiment of the suctioning/tracheostomy apparatus component of this system is depicted in FIG. 2. As depicted therein, in the suctioning/tracheostomy apparatus of the disclosed system the suctioning catheter and tracheostomy tube in combination are inserted into a patient's tracheostomy stoma (in the depicted embodiment, the suctioning catheter and trach tube enter a trach mask prior to entry to the trach stoma.) Accordingly, the suctioning/tracheostomy apparatus comprises an air column (301) having a proximal end (302) inserted into the tracheostomy stoma of a patient and a distal end (303) attached to an oxygen supply line (318) known to those of ordinary skill in the art, which is attached to an air compressor or other air providing device. The suctioning/tracheostomy apparatus also comprises a length of suctioning catheter (304) having a proximal end (305) inserted into the tracheostomy stoma of a patient and a distal end (306) attached to a suction canister (307) which is attached to a suction pump (not shown). As depicted in the FIG. 3, in this suctioning/tracheostomy apparatus, the suctioning catheter (304) is inserted into the tracheostomy stoma of a patient at the same time as the air column (301) (in the depicted embodiment through a trach mask).

While the suctioning/tracheostomy apparatus depicted in FIG. 2 is comprised of an air column (301) and suctioning catheter (304) combination it should also be understood that trach tubes known to those of ordinary skill in the art with lumens, perforations, internal channels, or other mechanisms which allow for suctioning of a trach tube without a secondary suctioning catheter are also contemplated for the air column (301) of the suctioning/tracheostomy apparatus. In these embodiments, the suctioning/tracheostomy apparatus will be comprised of an air column (301) with a proximal end (302) inserted into the tracheostomy stoma of a patient. The distal end (303) of the air column (301) will be attached to an air compressor. The air column (301) in this embodiment will also be attached to a suction pump in a manner known to those of ordinary skill in the art to create negative suctioning in the air column (301) when the patient's air column (301) needs to be suctioned. Thus, in this embodiment, suctioning is self-contained in the air column (301), no secondary catheter is required.

Further, notably, in some embodiments, it is contemplated that the suctioning/tracheostomy apparatus and the system described herein will be utilized with a patient on a ventilator. Cuffed trach tubes which create a vacuum and allow the ventilator to inflate the patient's lungs without air loss back through the trach opening are often associated with these patients. In certain embodiments, it is contemplated that the suctioning/tracheostomy apparatus and the system described herein may be used in conjunction with a mechanical ventilator to supply suctioning to a patient during the exhalation phase of ventilation. In these embodiments, it is contemplated that automated suctioning would occur as needed throughout the air column, including both above and below the cuffed trach.

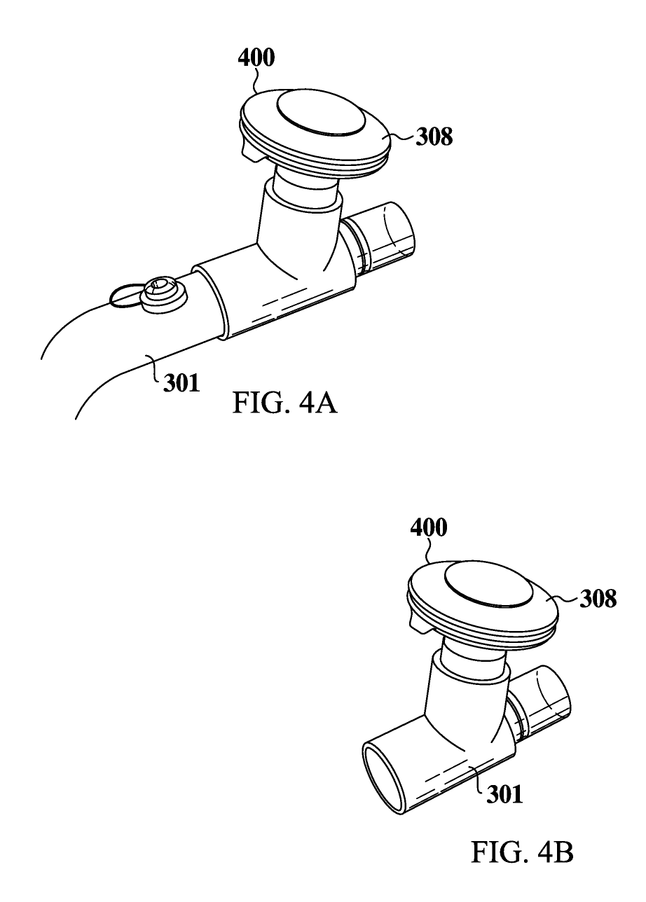

Another component of the suctioning/tracheotomy apparatus of the system is one or more microphones, transducers, sensors or other apparatuses known to those of ordinary skill in the art for converting sound into an electrical signal collectively referred to herein as “audio devices.” As depicted in FIG. 2, the audio device (308) of the suctioning/tracheotomy apparatus is located proximal to the length of air column (301) and generally connected to the length of air column (301) in such a manner so as to allow the audio device (308) to detect sound waves within the air column (301). The audio device (308) is generally attached to the air column (301) in such a manner as will be understood by those of ordinary skill in the art as to allow for the audio device (308) to detect a band of resonate audio frequencies within the tuned cavity created by the air column (301). One contemplated form of attachment of the audio device (308) to the air column (301) is shown in FIGS. 4a-b. In this embodiment, the audio device (308) is housed in a generally air-tight and waterproof housing (400) that is connected via a T-branch line to the air column (301). It is also contemplated, in alternative embodiments, that more than one audio device (308) may be utilized in the suctioning/tracheotomy apparatus. For example, more than one audio device (308) may be placed within the housing (400). Alternatively, more than one audio device (308) in separate housings (400) may be attached along the length of the air column (301). No matter the form of attachment to the air column (301) it is contemplated that the audio device (308) will be attached to the air column (301) in such a manner that it can continuously function in a moisture laden environment.

In one embodiment, the audio device (308) will be able to detect frequencies from 30 Hz to 3,000 Hz within the air column (301). This ability of the suctioning/tracheotomy apparatus to detect a range of frequencies is exploited by the system to monitor the breathing of a patient, detect any fluid obstructions, detect belabored breathing caused by a constricted airway, remove any fluid obstructions by activating a suction pump as needed and dispense a bronchodilator as needed. Each of these functions is generally performed without the assistance of a caregiver. In one embodiment, the audio device (308) will be connected to the rest of the system via an input module known to those of ordinary skill in the art. The input module will be interfaced to a processor, such as the field programmable gate array processor, for monitoring by the system.

In an alternative embodiment, another component of the suctioning/tracheotomy apparatus is a nebulizer (309) as shown in FIG. 2. A nebulizer, as that term is used herein, includes any device known to those of ordinary skill in the art to administer medication in the form of a mist inhaled into the lungs. The nebulizer (309) is located proximal to the length of air column (301) and is generally connected to the length of air column (301) in a manner known to those of ordinary skill in the art for administering a medication to a patient through the length of air column (301). In one embodiment, the nebulizer will be connected to the rest of the system via a digital output module by way of an external electronic relay. The digital output module, in turn, is interfaced to a processor (not shown) of the system, such as the field programmable gate array processor. In alternative embodiments, it is contemplated that multiple nebulizers may be attached to the air column (301). Further, in other alternative embodiments, it is contemplated that other components which can provide medicine, oxygen or other desired injectables to a patient are also contemplated components of the suctioning/tracheotomy apparatus.

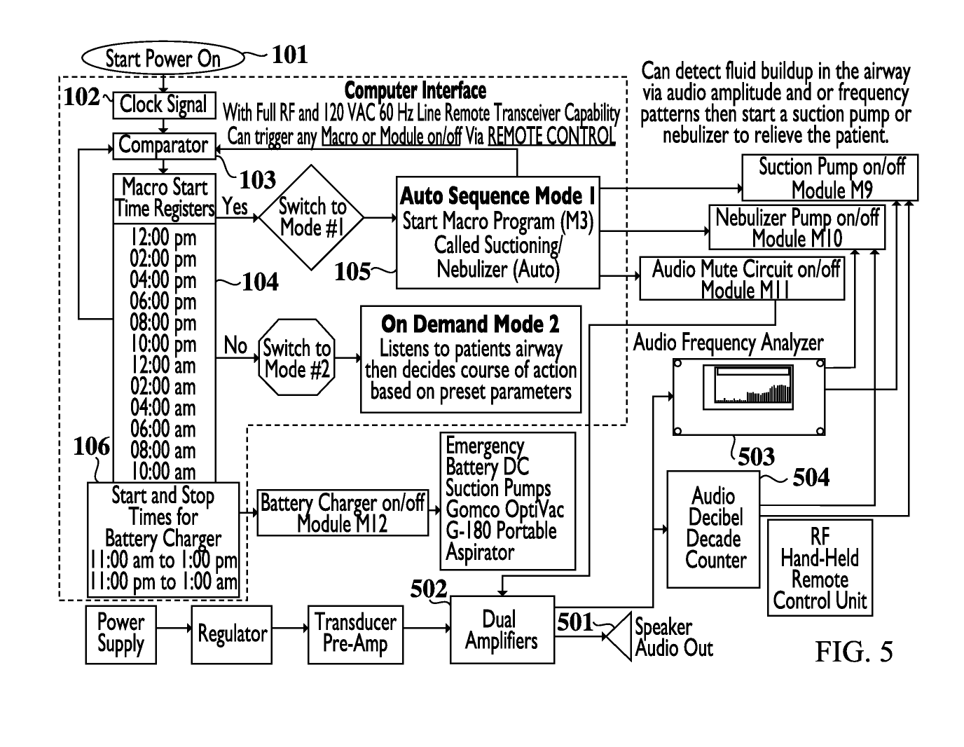

In general, the computer automated system for suctioning a patient, monitoring a patient and administering a prescribed dose of medication to a patient described herein has three (3) general modes of operation: the Auto Sequence Mode, the On Demand Mode and the Manual Mode. A flowchart which incorporates these modes of operation of the computer automated systems for suctioning a patient, monitoring a patient and administering a prescribed dose of medication to a patient is provided in FIG. 5. While the computer automated systems of this application are often described herein as having the automated functionality of both suctioning a patient, monitoring a patient, and administering a prescribed dose of medication, it should be understood that the computer automated systems can be programmed to automate only the suctioning of a patient in one embodiment, only the administration of a prescribed dose of medication in another embodiment, and only the monitoring of a patient in another embodiment, or various combinations of these functions.

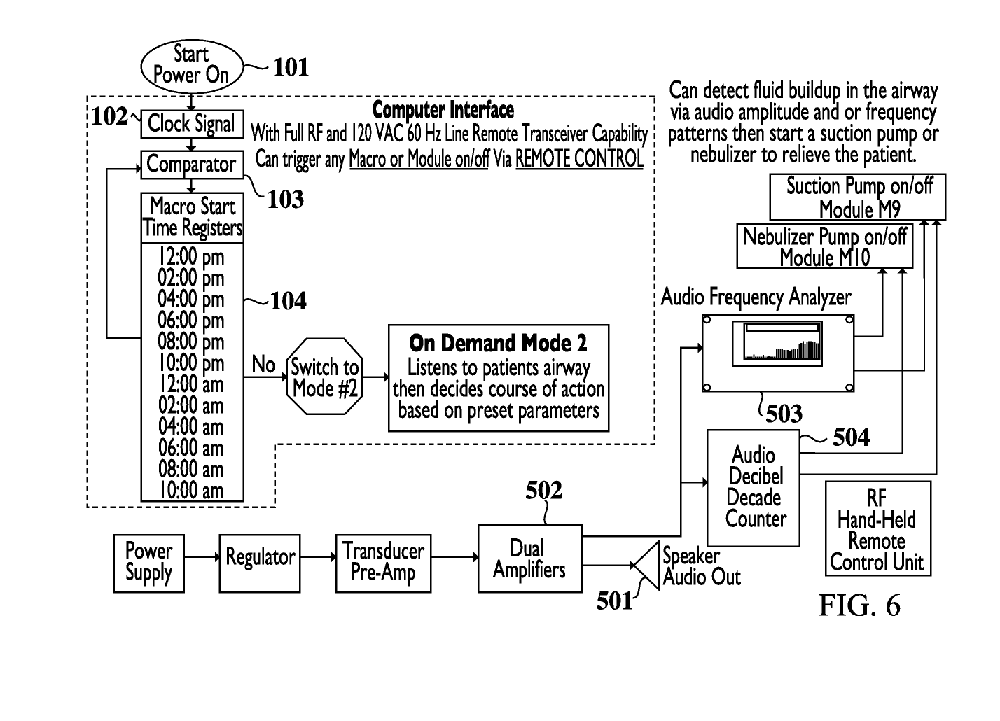

In the Auto Sequence Mode, a computer counts down a pre-set timing mechanism and triggers a software program which, at certain pre-defined intervals, suctions the patient and delivers a physician prescribed nebulized dose of medication to the patient. In one embodiment of the Auto Sequence Mode, the pre-set timing mechanism which the computer counts down is a 24-hour timing mechanism. In other embodiments however, this pre-set timing mechanism which the computer counts down may be a 12-hour timing mechanism, a 6-hour timing mechanism, or any other interval timing mechanism. Further, in one embodiment of the Auto Sequence Mode, depicted in FIG. 6, the software program will be triggered at two (2) hour intervals; however, it should be understood that any period of time is contemplated in this application as possible pre-defined intervals at which the software program is triggered.

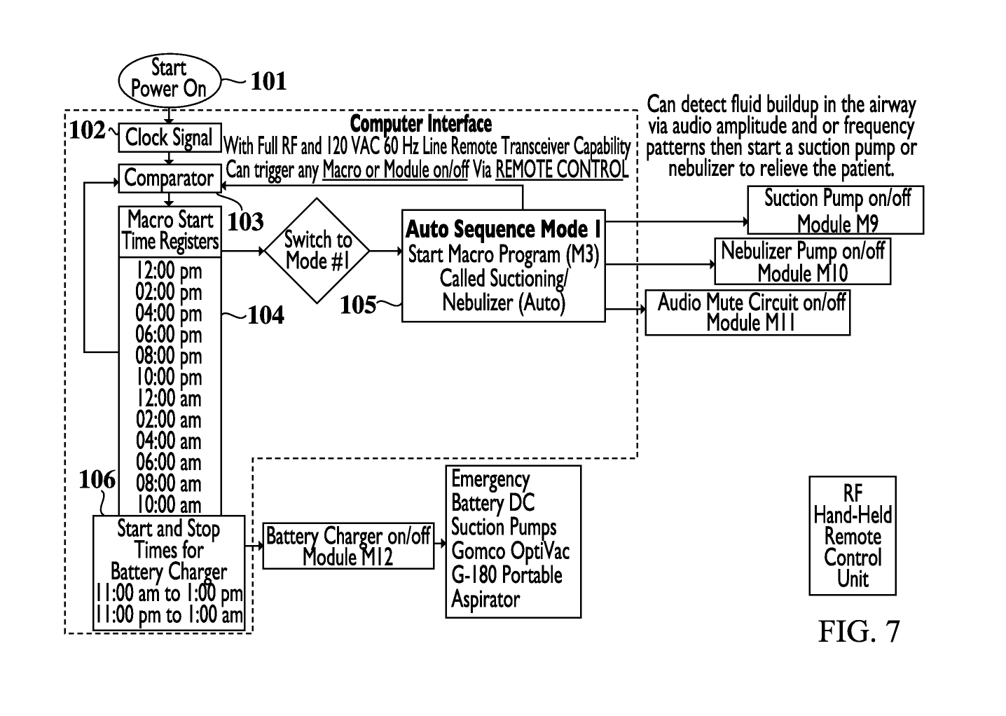

An embodiment of the Auto Sequence Mode is depicted in the flow chart of FIG. 7. In this embodiment, in a first step (101), the computer automated system for suctioning a patient and administering a prescribed dose of medication to a patient is turned on. In a second step, (102), a clock signal is checked. In a third step (103), the real time is compared to a pre-set start time for the Auto Sequence Mode to determine if the pre-set start time has been reached. In a fourth step (104), the software program engages at certain pre-determined interval time periods, such as every two (2) hours. When engaged, the software program will start its suctioning and nebulizing cycles for a fixed period of on and off cycles (105). The length of the suctioning and nebulizing cycles can be determined by the user. For example, in one embodiment the user can set the system to operate in three (3) ten (10) second suctioning cycles with twenty (20) to thirty (30) second “rests” between each cycle followed by a nebulizing cycle of thirty (30) seconds. Once completed, the program will return to its normal state, waiting until the end of the next predetermined time period at which point the software program will engage again.

In certain embodiments, as depicted in FIG. 7, it is contemplated that the Auto Sequence Mode will contain instructions for time intervals for engagement of a battery charger (106). This allows for the battery charger to automatically charge at certain predetermined time intervals. Aspects of the system as a whole which could be charged by a battery during these charging periods include, but are not limited to, suction pumps, vacuums and aspirators.

In sum, the software program of the Auto Sequence Mode generally functions to suction the patient and deliver a nebulized dose of medication by turning on and off a suction pump and a nebulizer in a sequence of pre-set time on and rest cycles.

For exemplary purposes, in one particular embodiment of the Auto Sequence Mode, when engaged, the suctioning apparatus for the air column is instructed to turn on for 30 seconds and then, subsequently, is instructed to turn off for 20 seconds. Next, the suctioning apparatus is instructed to turn on again for 30 seconds, followed by another 20 second rest cycle. After these two cycles of 30 second suctioning/20 second rest, the software program turns on the nebulizer for 30 seconds, followed by 20 seconds of rest. This is followed by another 30 second suctioning/20 second rest cycle. Then the Auto Sequence Mode turns on the nebulizer again for 30 second, followed by a 20 second rest cycle. Then, again, the suction mechanism is turned on for a 30 second cycle. After the completion of this 30 second suctioning cycle, and after a delay, the software program disengages.

Notably, while this described embodiment of the software program has specifically defined pre-set time on and rest cycles, any combination of time on and rest cycles for the suctioning apparatus and the nebulizer is contemplated in this application. Furthermore, it shall be understood that the Auto Sequence Mode can be programmed to simply control the functioning of the suctioning apparatus in certain embodiments. Likewise, the Auto Sequence Mode can be programmed to simply control the functioning of the nebulizer in certain embodiments.

While in the Auto Sequence Mode, it is contemplated that the engagement and disengagement of the suctioning apparatus, nebulizer and battery charger will occur automatically through the computerized methodology of the software program. It will also be possible, in certain embodiments, to engage and disengage the suctioning apparatus, nebulizer and battery charger directly through the Manual Mode. Depending upon the embodiment, this engagement of the Manual Mode can occur via the user interface or a remote control apparatus known to those of ordinary skill in the art. Through the remote control unit, user interface or other methodology known to those of ordinary skill in the art for interacting with a computer system, a user can directly engage or disengage the suctioning apparatus, nebulizer and battery charger of the computer automated system for suctioning a tracheostomy patient, monitoring a patient and administering a prescribed dose of medication to a tracheostomy patient. Stated differently, it allows a user to turn on or off these particular apparatuses outside of their automated on and off periods in the Auto Sequence Mode.

The third mode of the computer automated systems for suctioning a patient, monitoring a patient and administering a prescribed dose of medication to a patient is the On Demand Mode. In the On Demand Mode, the computer automated system monitors a patient's breathing via detection of a band of resonant audio frequencies within the tuned cavity of the air column. In certain embodiments, this is accomplished through an audio device that can detect secretion build-up through gagging or choking noises (by monitoring the audio amplitude and frequency patterns of such noises) and, when such noises are detected, start a suction pump to relieve the patient. Stated differently, the On Demand Mode listens to patient's airway noises through an audio device coupled to the air column and decides a course of action based upon preset parameters. Notably, in certain embodiments it is contemplated that the system will be configured in such a manner so as to be able to distinguish sounds while in the On Demand Mode; i.e., it will be able to filter out sounds other than the patient's breathing noises which are present in the system. As previously noted, contemplated noise cancellation techniques include digital filtering, active noise cancellation, linear regression analysis, principal component analysis, orthogonal decomposition, acoustical analysis of a patient's signal and other known techniques and mathematical algorithms for isolating a certain noise from other sounds present in the system. Such sounds include, but are not limited to, the ambient noise created by the air compressor, nebulizer and suction pump, among others.

One embodiment of the On Demand Mode is provided in FIG. 6. In this embodiment of the On Demand Mode, the On Demand Mode is engaged in the time periods when the periodic suctioning/nebulizing events triggered by the pre-set time intervals of the Auto Sequence Mode are not engaged. i.e., while the Auto Sequence Mode is between the pre-set intervals. When engaged, an apparatus for determining the presence of noise (referred to as an audio device herein), including but not limited to a microphone, an audio transducer, a speaker or another suitable apparatus for the detection of noise known to one of ordinary skill in the art, is turned “on”; i.e., it is in a mode in which it can detect the presence of noise or other audio patterns within the tuned cavity created by the air column (301).

Next, any noise picked up by the audio device is then, in certain embodiments, amplified by an amplifying system known to those of ordinary skill in the art for amplifying decibels (502). Once amplified, the noise is transmitted to an audio frequency analyzer (503), an audio decibel counter (504), or other device known to those of ordinary skill in the art for detecting a certain audio frequency. These devices detect the audio output from the audio device. Once the audio output reaches a certain pre-set level, also known as the choke threshold, on the audio frequency analyzer (503), the audio decibel counter (504), or other comparable device, will trigger the engagement of either or both the suction pump and/or the nebulizer. This choke threshold or trigger point can be adjusted for each individual patient and their respective needs with the system. The suction pump and the nebulizer will continue to stay “on” in this mode for a predetermined set period of time. In one embodiment, the predetermined set period of time will be 20 seconds. At the end of this set period of time, the suction pump and/or nebulizer will shut off unless the audio output is still above the pre-set choke threshold level on the audio frequency analyzer (503), audio decibel counter (504), or other comparable device. In this instance, the suction pump and/or nebulizer will continue to run until the noise level is below the pre-set choke threshold level on the audio frequency analyzer (503), the audio decibel counter (504), or other comparable device. Thus, as long as the patient's airway is blocked (signaled by the high decibel level in the air column), the suction pump and/or nebulizer will continue to stay on.

In certain embodiments, the choke threshold will be adjustable through the user interface to allow a user to modify the audio decibel at which the suction pump and/or nebulizer are engaged in the On Demand Mode to adjust the system to the sensitivities of a patient.

Further, in other embodiments, it is contemplated that there will be an auto mute function associated with the On Demand Mode. This auto mute function turns off or mutes the audio device of the On Demand Mode. This function can be used to completely mute the audio device (which is useful, e.g., when a patient is being moved so that they can be manipulated without triggering the system) or to simply mute the audio device when the computer automated system is in the Auto Sequence Mode. Similar to the suction pump, nebulizer and battery charger, it is contemplated that the auto mute function can be engaged or disengaged directly through a remote control device, the user interface or other methodology known to those of ordinary skill in that art and as described previously in this application.

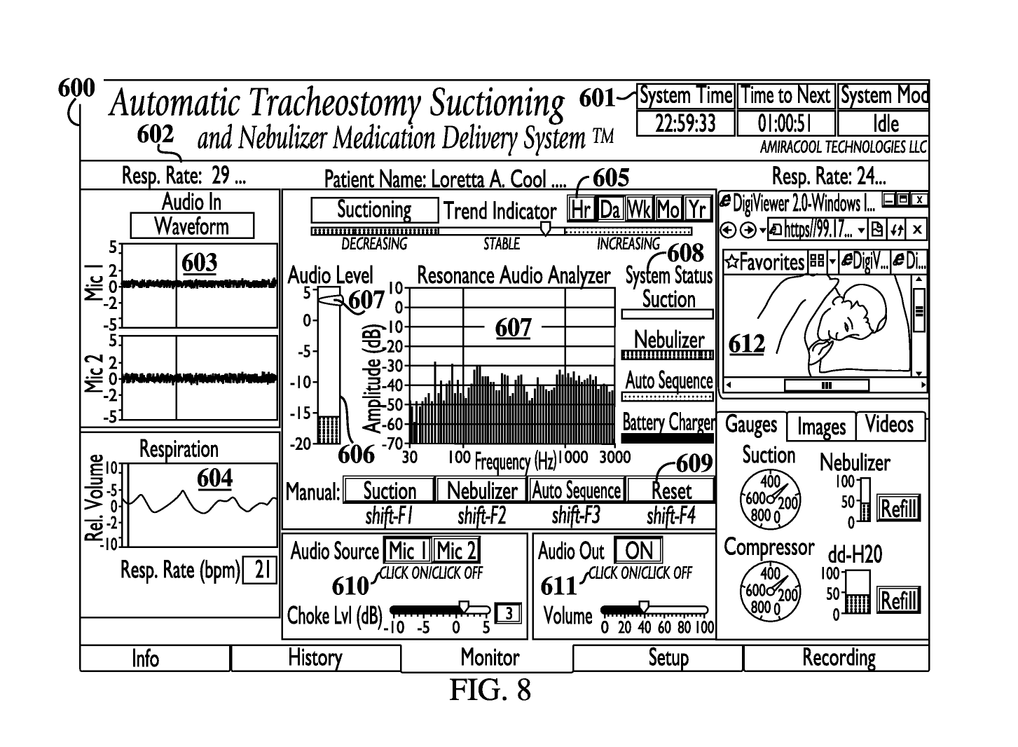

To further the understanding of how a user engages with the disclosed computer system, FIGS. 8-12 provide depictions of contemplated embodiments of the user interface for the disclosed computer system. It should be noted that none of these interface designs are determinative and that any possible interface design that allows a user to monitor the patient's vital signs, choke threshold, respiration and the operation of the disclosed computer system is contemplated in this application.

In one embodiment, the interface depicted in FIG. 8, the Monitor Interface (600), will be the default interface provided on the personal computer for the user. Among other displays, in certain embodiments it is contemplated that the monitor interface will display the following to a user of the system. First, the Monitor Interface (600) may include a status bar (601). The status bar (601) will generally include information regarding the general status of the system such as, but not limited to, the time, the period of time until the next suction/nebulizing cycle in the Auto Sequence Mode, and the current mode of the system. The current mode of the system notifies the user whether the system is suctioning, nebulizing, running idle or taking some other action. Second, the Monitor Interface (600) may include a scrolling notification bar (602). It is contemplated that this scrolling notification bar (602) will include information about the patient and information pertinent to patient care. Such information includes, but is not limited to, the patient's name, the patient's respiration rate, patient identifying numbers and the nurse or doctor in charge of the patent, among other pertinent information. Third, the Monitor Interface (600) may include audio device spectrograms (603). These spectrograms offer a time-varying spectral representation that depict in a graphic nature the degree with which the sounds picked up by the audio device normalized for certain exterior noises (such as coughing or mucus build-up) vary over time.

Fourth, the Monitor Interface (600) may include respiration spectrograms (604). These spectrograms offer a graphical representation of a user's respiration volume over time, along with identifying the user's current respiration rate. Fifth, the Monitor Interface (600) may include a suctioning trend indicator (605). This trend indicator (605), in one embodiment using a slide mechanism, graphically depicts whether a patient's secretion build-up suctioning events in all modes (identified by information gathered in the system regarding the number of times suctioning was initiated by the presence of secretion build-up in the On Demand Mode) had increased, decreased or stayed the same over time. As depicted in FIG. 8, the time period for the dataset upon which the trend indicator is based can be changed. For example, the trend indicator can be modified to reflect how the patient's suctioning (and, by extension, secretion build-up) has changed over an hour, over the day, over the week, over the month, and over the year. Notably, these time variables are not determinative and can be modified to any known time variables in different embodiments of the trend indicator (605).

Sixth, the Monitor Interface (600) may include an audio level indicator (606). The audio level indicator (606), through a graphical interface, depicts the current decibel level, after neutralization of ambient noise, that is detected by the system. In one embodiment of the audio level indicator (606), the indicator (606) will include a moveable marker (607) which represents the choke threshold at which the On Demand Mode will be activated. Seventh, the Monitor Interface (600) may include a resonance audio analyzer (607). This resonance audio analyzer graphically depicts the noise detected by the audio device (308), after neutralization of ambient noise, as a function of the amplitude of the noise, measured in decibels, over the frequency of the noise measure in Hz. Eighth, the Monitor Interface (600) may include a system status indicator (608). This indicator notifies a user whether the system is suctioning, nebulizing, in the Auto Sequence Mode and/or charging its battery. Ninth, the Monitor Interface (600) may include a control panel (609) for the Manual Mode discussed previously in this application. This control panel (609) provides an interface through which a user can manually manipulate the system. For example, in the depicted embodiment, a user can manually activate suctioning, nebulizing, or the Auto Sequence Mode through the control panel (609). In another embodiment, the control panel will also provide a means through which a user can reset the control panel (609).

Tenth, the Monitor Interface (600) may include an audio source display (610). This display will provide another interface through which the user can modify and alter the choke threshold level. In addition, this audio source display (610) will provide an interface through which a user can turn on or off the one or more audio devices (308) present in the system. Eleventh, the monitor interface may include an audio out display (611). This audio out display (611) lets a user choose whether or not the system transmits the audio noise captured by the system, such as the patient's breathing, via speakers or some other methodology for transmitting noise. Generally, this audio out display (611) will also include a mechanism through which a user can manipulate the volume of the audio released by the speakers. Twelfth, the Monitor Interface (600) may include a live video feed of the patient (612). Thirteenth, the Monitor Interface (600) may include a gauge display (613). This gauge display (613) provides an interface for a user to ascertain, amongst other things, the level of the suction pump when engaged, the level of the compressor when engaged, the amount of fluid left in the nebulizer, and the amount of fluid left in the sterile water container.

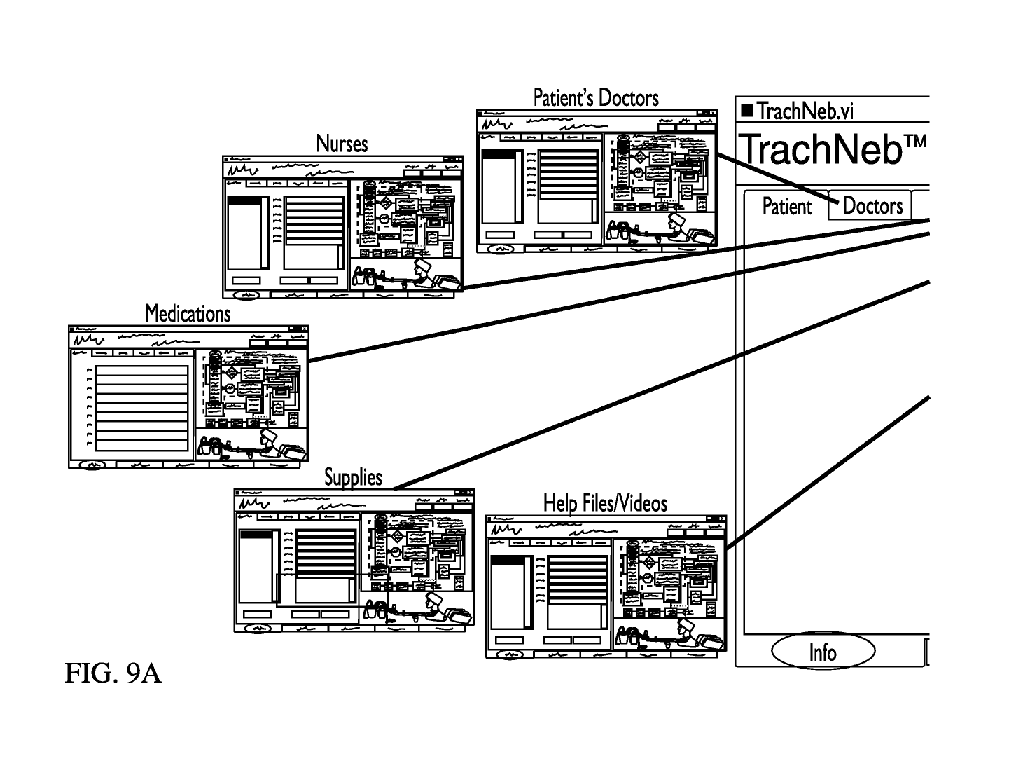

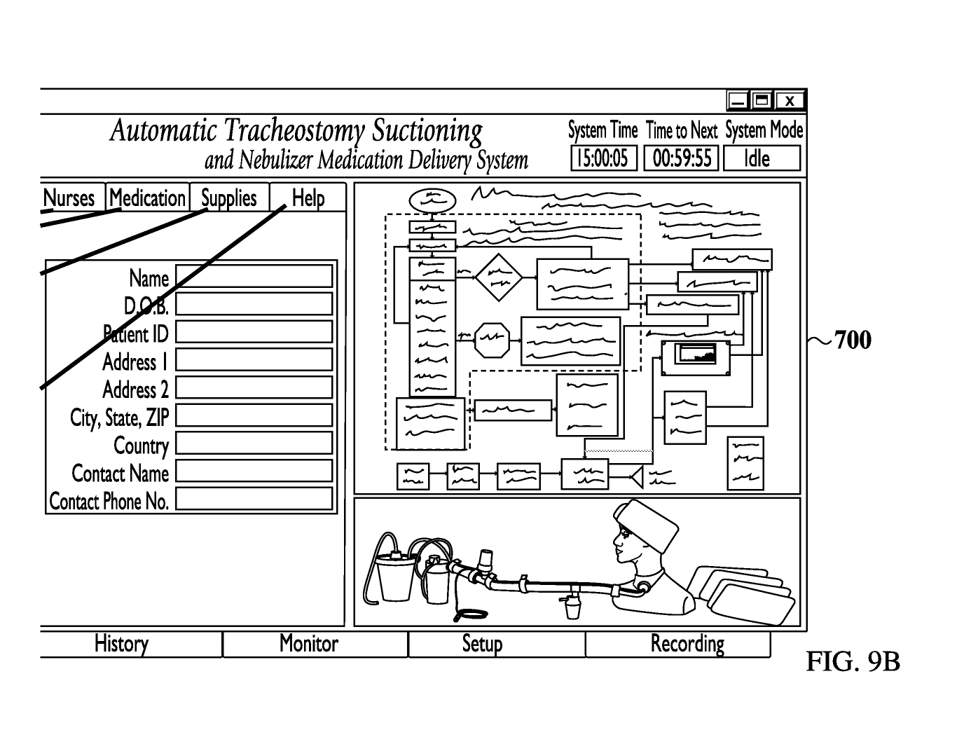

Another contemplated interface in the system is the Informational Interface (700) depicted in FIGS. 9a-b. In general, the Informational Interface provides an interface through which a user can easily access and modify pertinent information regarding the patient and the system. Such information includes, but is not limited to, biographical patient information (e.g., name, D.O.B., patient ID, patient address, and emergency contact information), information regarding the patient's doctors, information regarding the patient's nurses, information regarding the patient's medications, information regarding supplies needed by the patient during their care, help videos and files regarding operation of the system and other information pertinent to patient care and proper operation of the system.

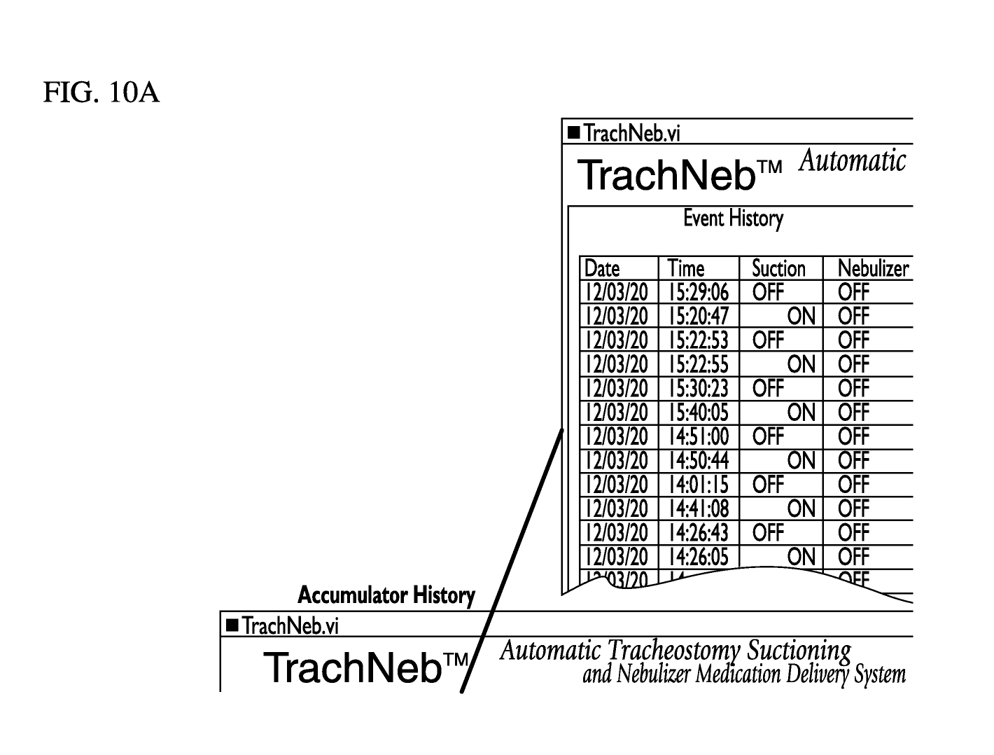

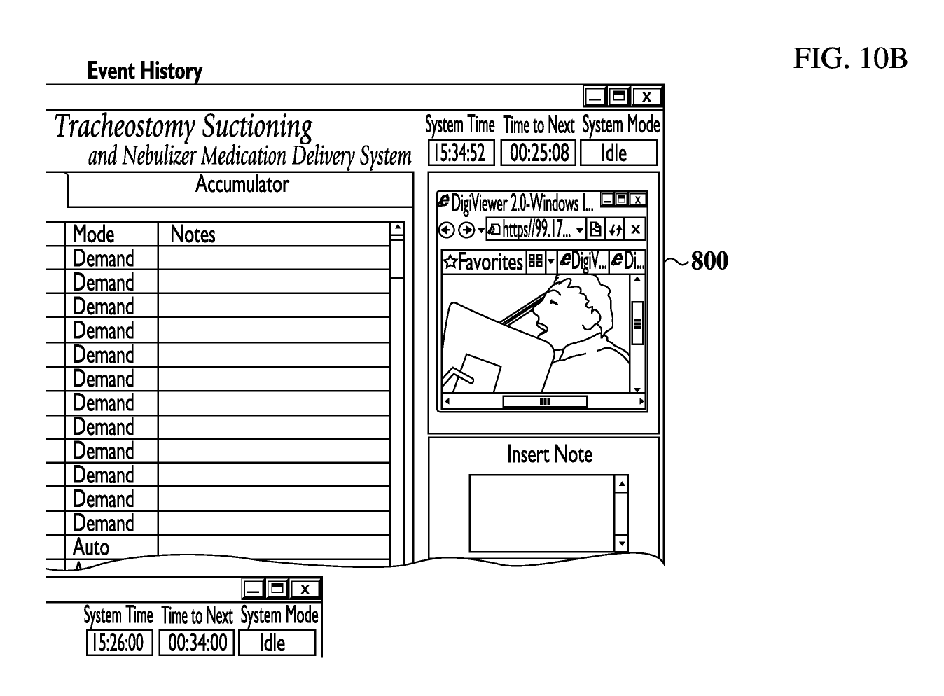

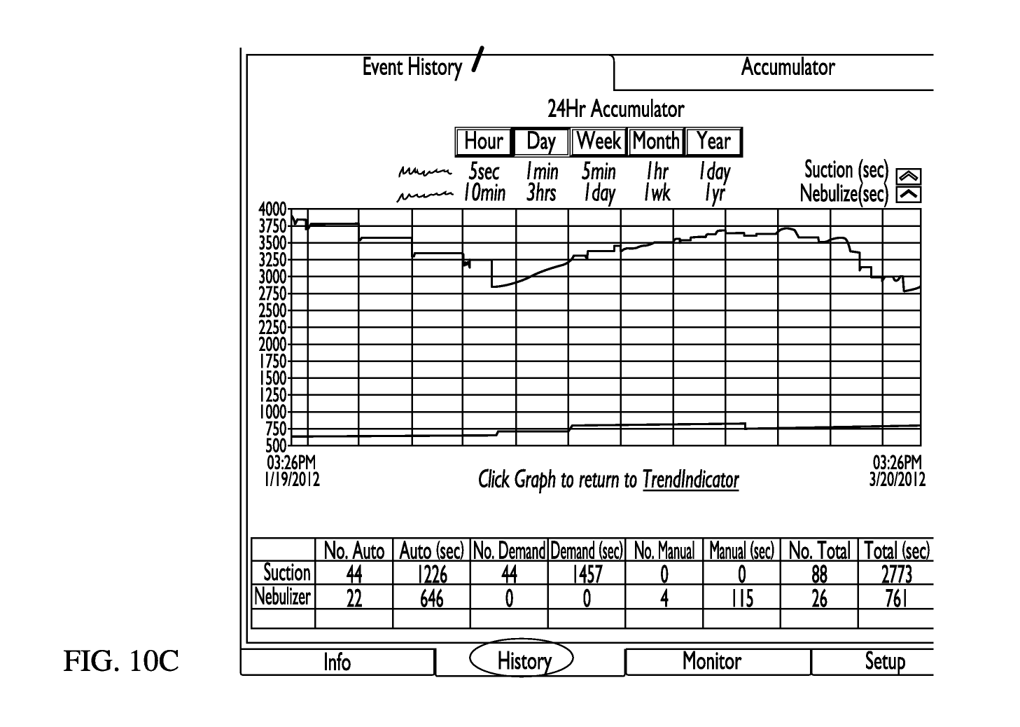

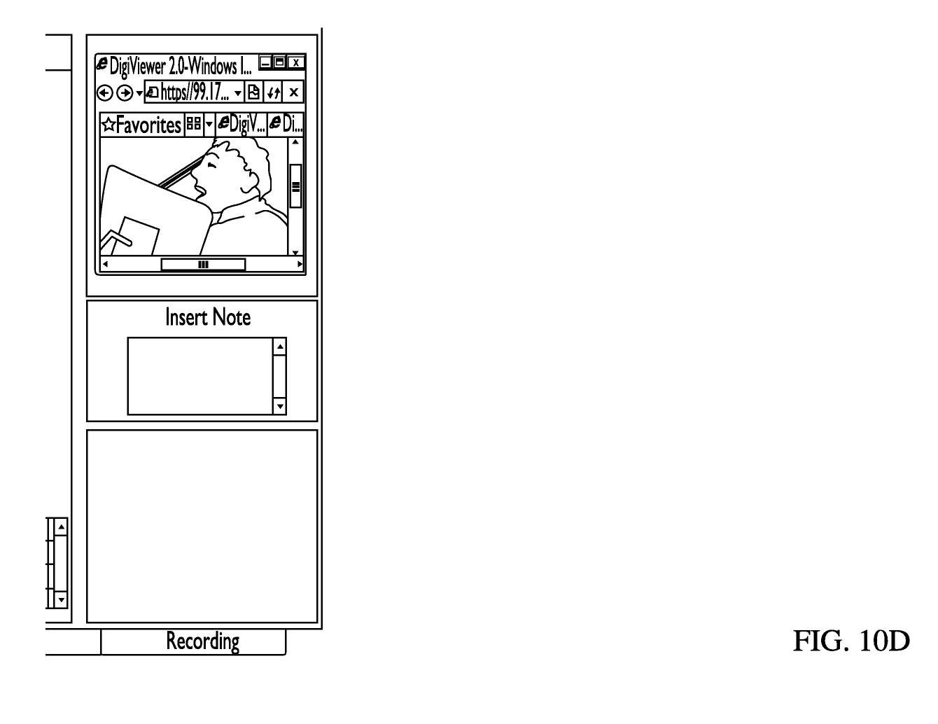

Another contemplated interface in the system is the History Interface (800), an embodiment of which is depicted in FIGS. 10a-d. Among other things, this History Interface (800) provides the user access to the event history database for the system; i.e., a database that compiles the suctioning and nebulizing events for the system. Information compiled in this database can include, but is not limited to, the date, time, and associated modes for different suctioning and nebulizing events in the system's history. In an embodiment, this History Interface (800) will also provide a graphical accumulator which will depict how the patient's suctioning and nebulizing has changed over a chosen period of time (e.g., an hour, day, week, month or year). In certain embodiments, the History Interface (800) will also include a data input section in which a user can insert notes regarding certain suctioning and nebulizing events in the patient's care history.

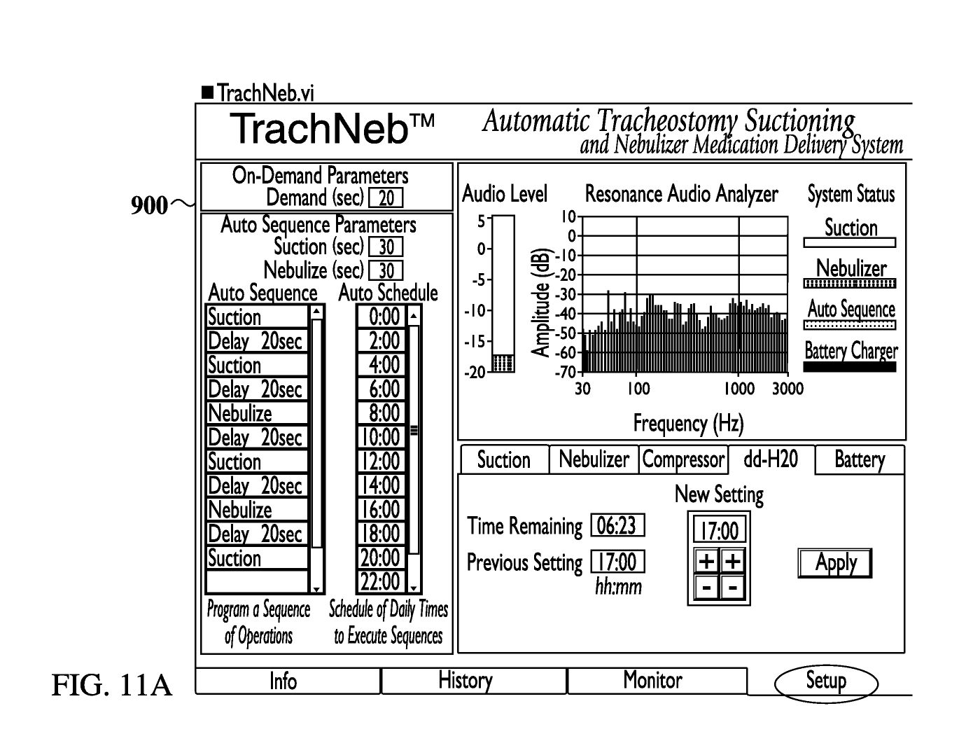

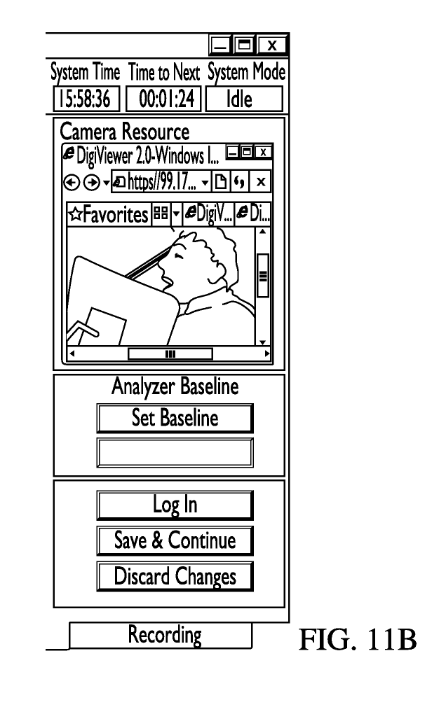

Yet another contemplated interface is the Setup Interface (900), an embodiment of which is depicted in FIGS. 11a-b. This interface will provide means through which a user can manipulate and modify the parameters of the system. For example, the user will be able to modify the suctioning time period in the On Demand Mode and the suctioning, delay and nebulizing periods in the Auto Sequence Mode. Further, in certain embodiments, a user will be able to modify certain desired settings for the suction, nebulizer, compressor, deionized water and battery.

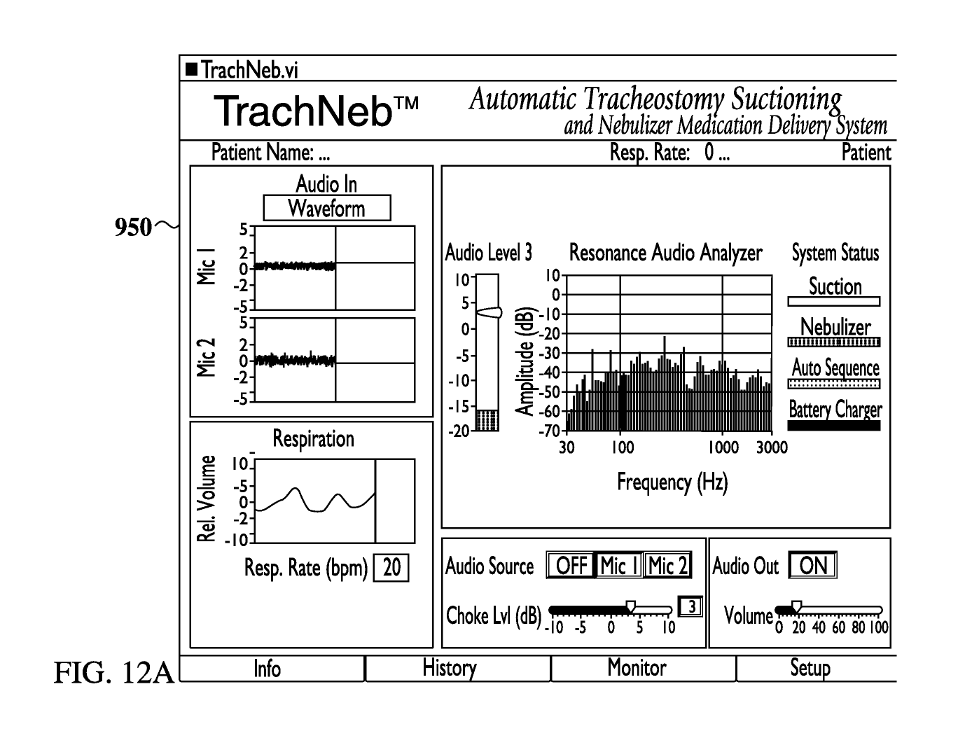

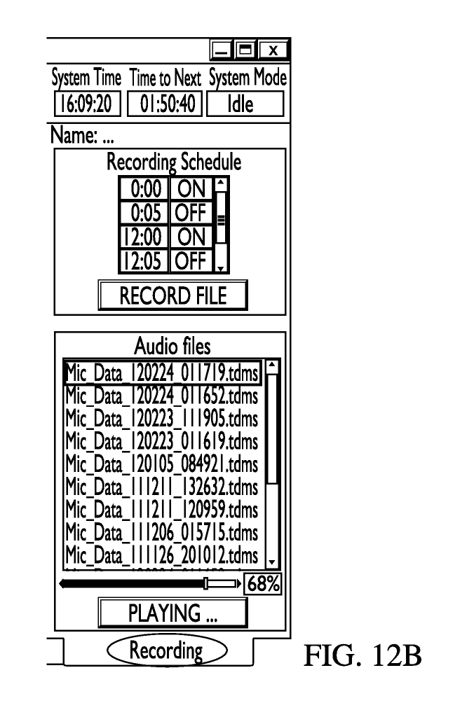

Another contemplated interface for the system is the Recording Interface (950), an embodiment of which is depicted in FIGS. 12a-b. This interface will allow a user access to past audio files generated by the system and allow a user to set a recording schedule for the system. In certain embodiments, such as that depicted in FIGS. 12a-b, this interface may also include the audio spectrogram, respiration spectrogram, resonance audio analyzer, audio source display and audio out display which may also be include in the Monitor Interface (600). Generally, any data or graphical interface regarding the audio or visual files created by the system may be accessed or manipulated via this interface.

In another embodiment of the system, it is contemplated that the system will have the capability of monitoring the fluid content in the nebulizer. In one embodiment, the system will be able to modify the fluid content in the nebulizer from time calculations. For example, the system will know the initial capacity of the nebulizer, the amount of fluid used per nebulizing event, and the number of nebulizing events since the last refill of the nebulizer. From this information, in one embodiment, the system will be able to determine the real-time fluid content of the nebulizer. Accordingly, the system will be able to notify a user when the fluid levels in the nebulizer are getting low and, accordingly, need to be refilled.

In addition to monitoring the fluid level of the nebulizer, in alternative embodiments it is contemplated that the system will also be able to monitor and analyze the efficacy of medication and the dosage amounts administered to a patient by the nebulizer. For example, the database of the system will keep information regarding: the number of times the patient had been nebulized across a certain time period and the number of incidences of suctioning initiated in the On Demand Mode. It is contemplated that, from this information, certain embodiments of the system will be able to present to the user via the interface the efficacy of a certain medication administered by the nebulizer over time.

It should also be recognized that the system described herein also provides a new methodology for monitoring the respiratory rate and other vital signs of a patient. Instead of ascertaining a patient's respiratory rate and breathing through the often unreliable and imprecise thoracic transducer belt and electrocardiograph methodologies that are currently the standard of care, the system described herein, through the audio device (308) coupled to the air column (301), is able to monitor and track a patient's breathing through audible means; i.e., via a patient's breathing pattern.

In addition, it is also contemplated in other embodiments that the system described herein will ascertain whether secretions or other impediments have built-up within a patient's airway and whether a patient's airway is constricted through visual, not audible means (or in one embodiment a combination of the both). In this embodiment, a fiber optic sensor, camera, or other technology known to those of ordinary skill in the art capable of determining the presence of an object in a certain defined area through visual means, will be placed on the proximal end (305) of the suctioning catheter (304) or within the air column (301) in the embodiment of the suctioning/tracheostomy apparatus without a separate suctioning catheter (304). This visual device will have the capability of detecting the presence or build-up of secretions within the air column (301). Thus, automatic suction of a patient and the automatic nebulizing of a patient will be initiated through visual detection of secretion build-up. In this embodiment of the system, in the On Demand Mode the suctioning of the patient will be initiated not when a certain decibel is reached but rather when a certain degree of secretion build-up is detected by the visual detection means within the air tubing (301). Thus, in this embodiment, the threshold will represent a certain visual level of detection of secretion within the air tubing (301) not a certain decibel level. Similarly, nebulizing of the patient with a bronchodilator will be initiated not when a certain decibel is reached, but rather when a certain degree of airway constriction is detected by the visual detection means.

In still further embodiments, it is contemplated that the system described herein will ascertain whether secretions or other impediments have built-up within a patient's airway through a sensor known to those of ordinary skill in the art that is capable of detecting moisture such as a hygrometer. In this embodiment, a moisture sensor known to those of ordinary skill in the art will be placed on the proximal end (305) of the suctioning catheter (304) or within the air column (301) in the embodiment of the suctioning/tracheostomy apparatus without a separate suctioning catheter (304). This moisture sensor will have the capability of detecting the presence or build-up of secretions within the air column (301). Thus, automatic suctioning of the patient will be initiated when a certain level of moisture is detected by the moisture sensor.

While the invention has been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of the invention.

1. A tracheostomy apparatus comprising:
an air column having a proximal and a distal end and a length there between, the proximal end being inserted into the tracheostomy opening of a patient and the distal end being connected to an oxygen supply line; and
at least one audio device connected to the length of air tubing;
wherein the at least one audio device receives audio frequencies including the patient's breath from the air tubing; and
wherein a processor interprets and responds to said audio frequencies.

2. The tracheostomy apparatus of claim 1 further comprising:
a length of suctioning catheter having a proximal end and a distal end, the proximal end being inserted into the tracheostomy opening of a patient and the distal end being attached to a suction pump.

3. The tracheostomy apparatus of claim 1 wherein the at least one audio device is capable of receiving frequencies from about 30 Hz to about 3,000 Hz.

4. The tracheostomy apparatus of claim 1 wherein the at least one audio device is chosen from the group consisting of: microphones, transducers and speakers.

5. The tracheostomy apparatus of claim 2 further comprising:
at least one nebulizer connected to the air column.

6. A non-transitory computer readable medium comprising:
computer readable instructions for counting down a pre-set timing mechanism; and
computer readable instructions for triggering the suctioning of an air column of a patient and the delivering of a nebulizing dose of medication by turning on and off a suction pump and a nebulizer in a sequence of pre-set time on and rest cycles.

7. A non-transitory computer readable medium comprising:
computer readable instructions for determining when noise detected within an air column of a patient has reached a certain pre-set level;
computer readable instructions for triggering the suctioning of the air column of a patient for a set period of time when the noise reaches the pre-set level.

8. The non-transitory computer readable medium of claim 9 further comprising:
computer readable instructions for triggering the continued suctioning of a patient at the end of the set period of time until the noise detected is below the certain pre-set level.

9. A method of automatically suctioning a patient, the method comprising:
setting a pre-set timing mechanism with certain pre-defined intervals;
counting down the pre-set timing mechanism; and
triggering the suctioning of a patient for a pre-set period of time when a certain pre-defined interval is reached.

10. A method of automatically suctioning a patient, the method comprising:
determining the level of noise within an air column to a patient;
determining when the level of noise within the air column reaches a certain pre-defined level;
triggering the suctioning of the air column for a pre-set period of time when the noise reaches the certain pre-defined level;
determining if the noise is still at the certain pre-defined level at the end of the pre-set suctioning time period; and
continuing the suctioning of the air column for another pre-set period of time if the noise is still at the certain pre-defined level.
